(19) United States
(12) Patent Application Publication
(10) Pub. No.: US 2009/0145434 A1
(43) Pub. Date: Jun. 11, 2009

(54) AROMATHERAPY FACE MASK AND METHOD THEREFOR

(76) Inventor: Kimberly J. Herrmann, Las Vegas, NV (US)

Correspondence Address: WEISS & MOY PC, 4204 NORTH BROWN AVENUE, SCOTTSDALE, AZ, 85251, US

(21) Appl. No.: 11/950,636

(22) Filed: Dec. 5, 2007

(51) Int. Cl. A61M 16/10; A61M 16/06

(52) U.S. Cl. 128/203.29

(57) ABSTRACT

A face mask, and specifically a surgical face mask, that uses aromatherapy to combat symptoms or improve the user's mood, and thoughts.

### FIELD OF THE INVENTION

This invention relates to face masks, and more specifically, to a face mask treated with essential oils to provide the wearer with the therapeutic benefits of aromatherapy.

### BACKGROUND OF THE INVENTION

The aroma of an essential oil is sensed by the olfactory nerve located in the back of the nose and carried to the brain, where it has its effect—perhaps stimulating or calming, perhaps imparting feelings of well-being and harmony to the whole self.

By incorporating aromatherapy into one's daily work routine, one can enjoy the natural health benefits of essential oils. These essential oils are concentrated essences of various flowers, fruits, herbs, and plants that have been used for centuries all over the world. Modern scientific research has proven that essential oils are potent, with remarkable medicinal properties. Unlike chemical drugs, essential oils do not remain in the body, and they do not leave toxins behind.

Disposable masks are used in many different settings, one of which is within a hospital setting. Such surgical face masks are intended for single-use purposes. However, none of the surgical face masks use aromatherapy. Therefore, a need exists for a face mask, and specifically a surgical face mask, that uses aromatherapy to combat symptoms or improve the user's mood, and thoughts.

### SUMMARY OF THE INVENTION

It is an object of the present invention to provide a face mask that uses aromatherapy to combat symptoms or improve the user's mood and thoughts.

### BRIEF DESCRIPTION OF THE PREFERRED EMBODIMENTS

In accordance with one embodiment of the present invention, an aromatherapy face mask is disclosed. The aromatherapy face mask comprises a rectangular shield comprising a fluid-resistant first layer, an absorptive second layer infused with aromatherapy essential oils, and a fluid-resistant third layer and two ear loops extending from opposite ends of the rectangular shield, each ear loop comprising a first end coupled to a top corner of the rectangular shield and a second end coupled to a bottom corner of the rectangular shield.

In accordance with another embodiment of the present invention, a method for using aromatherapy is disclosed. The method comprises the steps of providing a face mask comprising a face shield comprising a fluid resistant first layer, an absorptive second layer, and a fluid resistant third layer, two ear loops coupled at opposite ends of the face shield, infusing the second layer of the face shield with aromatherapy oils, wearing the face mask by a user, breathing in a scent of the aromatherapy oils by the user; and affecting a mood of the user with the scent of the aromatherapy oils.

In accordance with another embodiment of the present invention, a method for using aromatherapy in the health care profession is disclosed. The method comprises the steps of providing a surgical face mask comprising a face shield comprising a fluid resistant first layer, an absorptive second layer, and a fluid resistant third layer, two ear loops coupled at opposite ends of the face shield, infusing the second layer of the face shield with aromatherapy oils, wearing the face mask by a health care provider, breathing in a scent of the aromatherapy oils by the health care provider, and affecting the mood of the health care provider with the scent of the aromatherapy oils.

The foregoing and other objects, features, and advantages of the present invention will be apparent from the following, more particular, description of the preferred embodiments of the invention, as illustrated in the accompanying drawings.

### BRIEF DESCRIPTION OF THE DRAWINGS

FIG. 1 is a perspective view of an embodiment of the face mask of the present invention.

FIG. 2 is a cross-sectional view of the face mask of FIG. 1, taken along lines 2-2.

FIG. 3 is a perspective view of the face mask of FIG. 1 shown is use by a health care provider.

### DETAILED DESCRIPTION OF THE PREFERRED EMBODIMENTS

The novel features believed characteristic of the invention are set forth in the appended claims. The invention will best be understood by reference to the following detailed description of illustrated embodiments when read in conjunction with the accompanying drawings, wherein like reference numerals and symbols represent like elements.

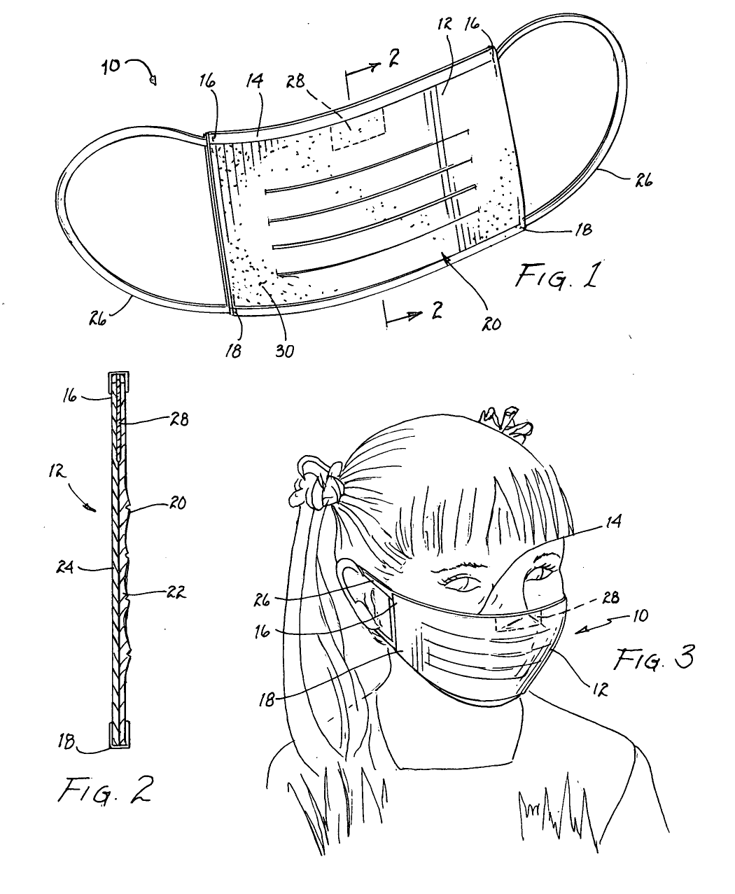

FIGS. 1-3 together refer to an aromatherapy face mask, hereinafter referred to as face mask 10. The face mask 10 has a face shield 12 that is infused with aromatherapy oils 30 and has two ear loops 26 coupled at opposite ends of the face shield 12.

FIG. 1 is a perspective view of an embodiment of the face mask 10 of the present invention. In this embodiment, the face shield 12 is rectangular and the two ear loops 26 extend from opposite ends of the rectangular face shield 12. Each ear loop 26 has a first end coupled to a top corner 16 of the rectangular face shield 12 and a second end coupled to a bottom corner 18 of the rectangular face shield 12. It should be clearly understood, however, that substantial benefit may be derived from the ear loops 26 being coupled to other suitable portions of the face shield 12. The face mask 10 may also have a moldable nose piece 28 coupled along a top edge 14 of the rectangular face shield 12.

Referring to FIG. 2, the face shield 12 is shown having three layers. The first layer 20 and the third layer 24 are both fluid-resistant. The second layer 22 is absorptive and is infused with essential aromatherapy oils 30 (see FIG. 1). Here, the nose piece 28 is shown being coupled between the second layer 22 and the third layer 24.

### Statement of Use

FIG. 3 shows the face mask 10 of FIG. 1 in use by a user 32. In this embodiment, the user 32 is a health care provider. However, it should be clearly understood that substantial benefit may be derived from a user 32 that is not a health care provider. The absorptive second layer 22 of the face shield 12 is infused with aromatherapy oils 30. The user 32 will wear the face mask 10 by placing the third layer 24 of the face shied 12 against his/her face and looping the ear loops 26 around the user's 32 ears. The user 32 will breathe in the scent of the aromatherapy oils 30 thus affecting the mood of the user 32.

The affect that the scent of the aromatherapy oils 30 have on the user 32 depends upon the scent of the oils 30. Some scents will affect the mood of the user 32. For example, vanilla scent will have a soothing and calming effect, sweet orange scent will promote happiness and reduce anxiety, grapefruit scent will induce euphoria and relieve performance anxiety, and chamomile will promote peace and reduce tension. Lemon scent will be refreshing and uplifting to the user 32. And a combination of lavender and vanilla scents will help combat stress and relieve tension.

Other scents may be useful in combating symptoms of the user 32. For example, peppermint scent may soothe a headache or clear the user's 32 sinuses. A combination of lemon, grapefruit, and geranium scents will activate and balance the mind and emotions, and may help clear the user's 32 head.

While the invention has been particularly shown and described with reference to preferred embodiments thereof, it will be understood by those skilled in the art that the foregoing and other changes in form and details may be made therein without departing from the spirit and scope of the invention.

I claim:

1. An aromatherapy face mask comprising:
a rectangular shield comprising:
a fluid-resistant first layer;
an absorptive second layer infused with aromatherapy essential oils; and
a fluid-resistant third layer; and
two ear loops extending from opposite ends of the rectangular shield, each ear loop comprising:
a first end coupled to a top corner of the rectangular shield; and
a second end coupled to a bottom corner of the rectangular shield.

2. The aromatherapy face mask of claim 1 further comprising a moldable nose piece coupled to a top edge of the shield.

3. The aromatherapy face mask of claim 1 wherein the aromatherapy essential oils have a scent of at least one of vanilla, peppermint, lemon, sweet orange, grapefruit, chamomile, lavender, citrus, eucalyptus, ginger, and geranium.

4. A method for using aromatherapy:
providing a face mask comprising:
a face shield comprising:
a fluid resistant first layer;
an absorptive second layer; and
a fluid resistant third layer;
two ear loops coupled at opposite ends of the face shield;
infusing the second layer of the face shield with aromatherapy oils;
wearing the face mask by a user;
breathing in a scent of the aromatherapy oils by the user; and
affecting a mood of the user with the scent of the aromatherapy oils.

5. The method of claim 4 wherein the scent of the aromatherapy oils provide a calming effect on the mood of the user.

6. The method of claim 5 wherein the aromatherapy oils have the scent of at least one of vanilla, orange, grapefruit, lavender, and chamomile.

7. The method of claim 4 wherein the scent of the aromatherapy oils provide an uplifting effect on the mood of the user.

8. The method of Clam 7 wherein the aromatherapy oils have the scent of at least one of lemon, orange, and grapefruit.

9. The method of claim 4 wherein the scent of the aromatherapy oils provide mental clarity to the user.

10. The method of claim 9 wherein the aromatherapy oils have the scent of at least one of lemon, grapefruit, and geranium.

11. The method of claim 4 further comprising the step of combating symptoms of the user.

12. The method of claim 11 wherein in the aromatherapy oils have the scent of at least one of peppermint, lemon, ginger, eucalyptus, and lavender.

13. A method for using aromatherapy in the health care profession:
providing a surgical face mask comprising:
a face shield comprising:
a fluid resistant first layer;
an absorptive second layer; and
a fluid resistant third layer;
two ear loops coupled at opposite ends of the face shield;
infusing the second layer of the face shield with aromatherapy oils;
wearing the face mask by a health care provider;
breathing in a scent of the aromatherapy oils by the health care provider; and
affecting the mood of the health care provider with the scent of the aromatherapy oils.

14. The method of claim 13 wherein the scent of the aromatherapy oils provide a calming effect on the mood of the health care provider and wherein the aromatherapy oils have the scent of at least one of vanilla, orange, grapefruit, lavender, and chamomile.

15. The method of claim 13 further comprising the step of combating symptoms experienced by the health care provider.

16. The method of claim 15 wherein combating symptoms experienced by the health care provider comprises the steps of:
stimulating the immune system of the health care provider; and
providing for healthy respiration for the health care provider; and
wherein the aromatherapy oils have the scent of eucalyptus and lavender.

17. The method of claim 15 wherein combating symptoms experienced by the health care provider comprises the step of treating nausea experienced by the health care provider and wherein the aromatherapy oils have the scent of lemon and ginger.

18. The method of claim 15 wherein combating symptoms experienced by the health care provider comprises the steps of:
clearing the head of the health care provider; and
balancing the mind and emotions of the health care provider;
and wherein the aromatherapy oils have the scent of lemon, grapefruit, and geranium.

19. The method of claim 15 wherein combating symptoms experienced by the health care provider comprises the step of soothing a headache of the health care provider and wherein the aromatherapy oils have the scent of peppermint.

20. The method of claim 15 wherein combating symptoms experienced by the health care provider comprises the step of clearing the sinuses of the user and wherein the aromatherapy oils have the scent of peppermint.
